(19) United States
(12) Patent Application Publication
(10) Pub. No.: US 2025/0253057 A1
(43) Pub. Date: Aug. 7, 2025

(54) PREDICTING DISEASE PROGRESSION IN PORTAL HYPERTENSION USING MACHINE LEARNING

(71) Applicant: ASTRAZENECA AB, Södertälje (SE)

(72) Inventors: Nils SVANGÅRD, Södertälje (SE); Peter GREASLEY, Södertälje (SE); Philip AMBERY, Södertälje (SE); Shameer KHADER, Wilmington, DE (US)

(21) Appl. No.: 18/855,064

(22) PCT Filed: May 18, 2023
(86) PCT No.: PCT/IB2023/000296

§ 371 Date: Oct. 8, 2024

Related U.S. Application Data
(60) Provisional application No. 63/346,189, filed on May 26, 2022.

(51) Int. Cl. G16H 50/50; A61B 5/00

(52) U.S. Cl. CPC G16H 50/50; A61B 5/4842

(57) ABSTRACT

An example embodiment may involve obtaining an observation of demographic values, comorbidity values, vital sign values, and/or blood test values of an individual, wherein the individual was diagnosed with portal hypertension and/or cirrhosis; applying a machine learning model to the observation, wherein the machine learning model was trained with a training data set, wherein the training data set contains observations of corresponding demographic values, comorbidity values, vital sign values, blood test values, and/or disease progression values for a plurality of individuals diagnosed with portal hypertension and/or cirrhosis, and wherein the machine learning model is configured to provide a prediction of: (i) a hazard ratio of whether the individual is expected to exhibit progression to a condition related to portal hypertension or cirrhosis, and/or (ii) a period of time between that of the new observation and a further diagnosis of the condition; and providing the prediction based on the observation.

### CROSS-REFERENCE TO RELATED APPLICATION

This application claims priority to U.S. provisional patent application No. 63/346,189, filed May 26, 2022, which is hereby incorporated by reference in its entirety.

### BACKGROUND

The portal venous system supplies blood to the liver. Portal hypertension is elevated blood pressure in these veins, and is typically caused by cirrhosis (scarring of the liver) or thrombosis (clotting). In some cases, portal hypertension can lead to dilated veins in the esophagus and/or stomach (varices) that can bleed, resulting in life-threatening hemorrhages. Portal hypertension can also cause the accumulation of fluid in the abdomen (ascites). Beta blockers are the favored treatment for portal hypertension, but are only effective in controlling the condition in less than half of patients. Other therapies, such as liver shunts or liver transplants, are more invasive.

No large epidemiology studies have been published on portal hypertension for 20 years or more. Thus, the medical community's knowledge regarding the prognosis and clinical outcomes for portal hypertension patients is limited.

### SUMMARY

Portal hypertension typically progresses from mild cases, to cases of clinical significance, to more severe cases, and eventually death. However, different patients with portal hypertension progress differently and at different rates. Further, the types of treatments that may help a patient can be highly sensitive to not only the patient's current diagnosis but how their disease progresses. Thus, a diagnosis of portal hypertension alone does not provide a health care provider with sufficient information to make successful patient-specific treatment recommendations.

The embodiments herein involve developing, training, and using machine learning models to predict disease progression in portal hypertension patients. This predicted progression may indicate an outcome (e.g., varices, variceal hemorrhages, recurrent variceal hemorrhages, ascites, refractory ascites, hepatic encephalopathy, recurrent hepatic encephalopathy, and/or jaundice, to name a few). The predicted progression may also provide an expected time to reach that outcome. In this manner, patients at risk of complications and/or death can be identified earlier and more accurately. A health care provider can then make a more informed recommendation regarding a treatment regimen and the timing thereof.

Accordingly, a first example embodiment involves obtaining, by a computing system, a training data set, wherein the training data set contains observations of corresponding demographic values, comorbidity values, vital sign values, blood test values, and/or disease progression values for a plurality of individuals diagnosed with portal hypertension and/or cirrhosis; and applying, by the computing system, a machine learning trainer to the training data set, wherein the machine learning trainer produces a plurality of machine learning models, and wherein each of the machine learning models is configured to take a new observation of new demographic values, new comorbidity values, new vital sign values, and/or new blood test values as input and provide a prediction of: (i) a hazard ratio of whether an individual diagnosed with portal hypertension and/or cirrhosis exhibiting the new observation is expected to exhibit progression to a respective condition related to portal hypertension or cirrhosis, and/or (ii) a period of time between that of the new observation and a further diagnosis of the respective condition.

A second example embodiment involves obtaining, by a computing system, an observation of demographic values of an individual, comorbidity values of the individual, vital sign values of the individual, and/or blood test values of the individual, wherein the individual was diagnosed with portal hypertension and/or cirrhosis; applying, by the computing system, a machine learning model to the observation, wherein the machine learning model was trained with a training data set, wherein the training data set contains observations of corresponding demographic values, comorbidity values, vital sign values, blood test values, and/or disease progression values for a plurality of individuals diagnosed with portal hypertension and/or cirrhosis, and wherein the machine learning model is configured to provide a prediction of: (i) a hazard ratio of whether the individual is expected to exhibit progression to a condition related to portal hypertension or cirrhosis, and/or (ii) a period of time between that of the observation and a further diagnosis of the condition; and providing, by the computing system, the prediction based on the observation.

In a third example embodiment, an article of manufacture includes a non-transitory computer-readable medium, having stored thereon program instructions that, upon execution by a computing system, cause the computing system to perform operations in accordance with the first and/or second example embodiment.

In a fourth example embodiment, a computing system includes at least one processor, as well as memory and program instructions. The program instructions may be stored in the memory, and upon execution by the at least one processor, cause the computing system to perform operations in accordance with the first and/or second example embodiment.

In a fifth example embodiment, a system includes various means for carrying out each of the operations of the first and/or second example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a schematic drawing of a computing device, in accordance with example embodiments.

FIG. 2 illustrates a schematic drawing of a server device cluster, in accordance with example embodiments.

FIG. 3 illustrates the training and use of a gradient boosting model, in accordance with example embodiments.

FIG. 4 depicts progression paths for portal hypertension, in accordance with example embodiments.

FIG. 5 depicts an example timeline for progression of portal hypertension, in accordance with example embodiments.

FIG. 6 depicts two timelines for progression of portal hypertension or liver disease in general, in accordance with example embodiments.

FIG. 7 depicts training and use of an array of machine learning models, in accordance with example embodiments.

FIG. 8 depicts use of a trained machine learning model, in accordance with example embodiments.

FIG. 9 is a graph of area under the curve values establishing that the machine learning models described herein outperform conventional techniques, in accordance with example embodiments.

FIGS. 10 and 11 are flow charts, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein. r embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. EXAMPLE COMPUTING DEVICES AND CLOUD-BASED COMPUTING ENVIRONMENTS

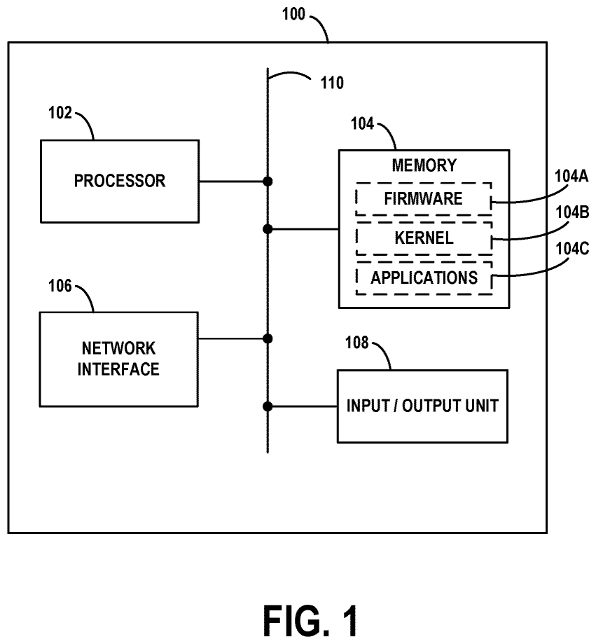

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently-used instructions and data.

Memory 104 may be any form of computer-usable memory, including but not limited to random access memory (RAM), read-only memory (ROM), and non-volatile memory (e.g., flash memory, hard disk drives, solid state drives, and/or tape storage). Thus, memory 104 represents both main memory units, as well as long-term storage. Other types of memory may include biological memory.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling, and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, and so on). Network interface 106 may also support communication over one or more non-Ethernet media, such as coaxial cables or power lines, or over wide-area media, such as Synchronous Optical Networking (SONET) or software-define wide-area networking (SD-WAN) technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

One or more computing devices like computing device 100 may be deployed to support the embodiments herein. The exact physical location, connectivity, and configuration of these computing devices may be unknown and/or unimportant to client devices. Accordingly, the computing devices may be referred to as “cloud-based” devices that may be housed at various remote data center locations.

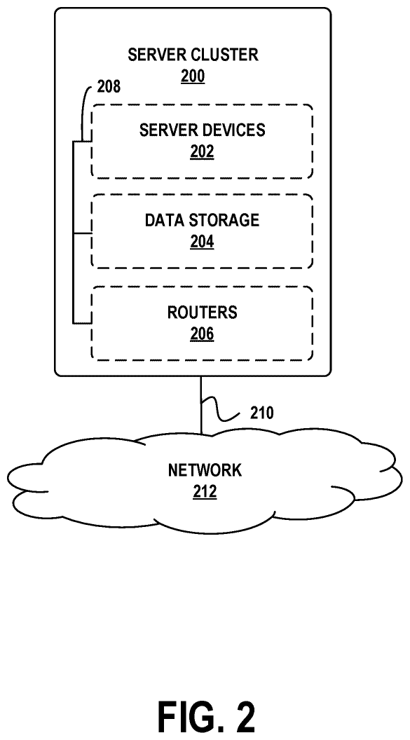

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purposes of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via local cluster network 208, and/or (ii) network communications between server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency, and/or other design goals of the system architecture.

As a possible example, data storage 204 may include any form of database, such as a structured query language (SQL) database. Various types of data structures may store the information in such a database, including but not limited to tables, arrays, lists, trees, and tuples. Furthermore, any databases in data storage 204 may be monolithic or distributed across multiple physical devices.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page or web application representations, or for use by a software application in some other fashion. Such a representation may take the form of a markup language, such as HTML, the eXtensible Markup Language (XML), or some other standardized or proprietary format.

Moreover, server devices 202 may have the capability of executing various types of computerized scripting languages, such as but not limited to Perl, Python, PUP Hypertext Preprocessor (PUP), Active Server Pages (ASP), JAVASCRIPT®, and so on. Computer program code written in these languages may facilitate the providing of web pages to client devices, as well as client device interaction with the web pages. Alternatively or additionally, JAVA® may be used to facilitate generation of web pages and/or to provide web application functionality.

### II. EXAMPLE GRADIENT BOOSTING MODELS

Gradient boosting algorithms are machine learning techniques that can be used to develop prediction models for multi-dimensional data sets. For convenience, these sets are often represented in matrix form using columns and rows. One or more columns represent input variables, and a further column represents an output variable. The output variable is an unknown function of one or more of the input variables. The rows represent observations of input variables and their corresponding output variables, usually based on real-world data. In many cases, the number of rows can be quite large, in the hundreds, thousands, or more. The machine learning process involves training the gradient boosting model to be able to predict the output variable for new observations of the input variables. In other words, the model attempts to learn or at least approximate the unknown function from the existing instances of input variables and their corresponding output variables.

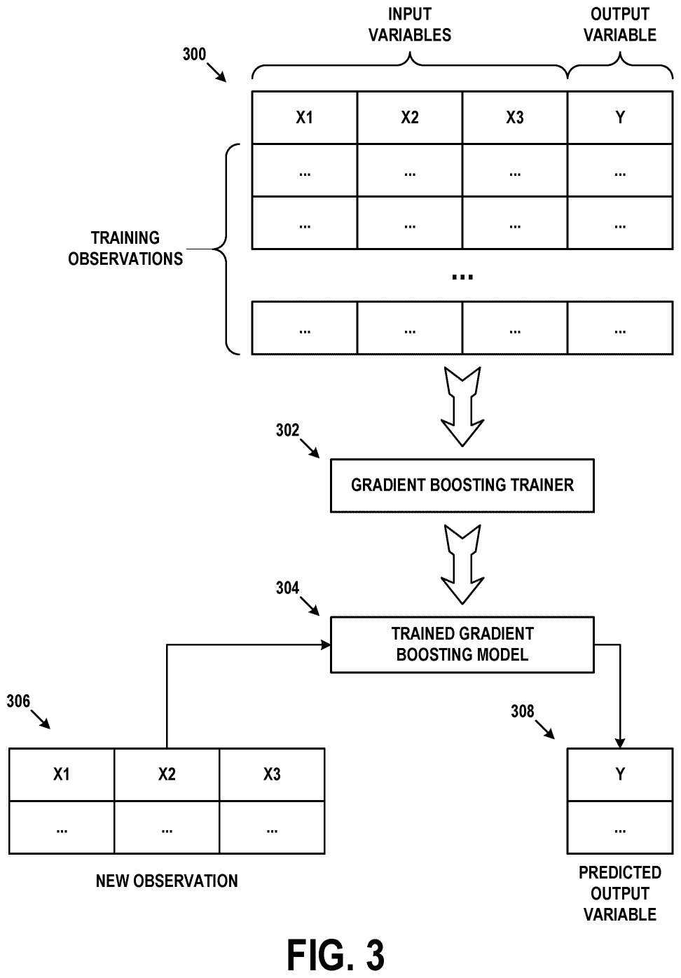

FIG. 3 further illustrates these concepts. Training data set 300 includes a set of training observations (rows), each consisting of input variables X1, X2, and X3 and their corresponding output variable, Y. These input and output variables are related by way of some unknown function ƒ, where Y=ƒ(X1, X2, X3). The output variable Y can take various forms, such as integer or real numbers, text, or Boolean values.

Training data set 300 may be gathered from actual patient medical data, e.g., from health professionals, hospitals, clinical trials or other sources. In such a training data set, the values of the output variable for each observation is expected to be known, but not every value of the input variables needs to be present—e.g., the training data set may be sparsely populated.

Training data set 300 is provided to gradient boosting trainer 302, which applies one or more training techniques to produce gradient boosting model 304. Gradient boosting model 304 may be an algorithm, or set of parameters to control the behavior of an algorithm, that can be used to apply an approximation of unknown function ƒ to new observations of the input variables.

Thus, gradient boosting model 304 may receive new observation 306 and produce predicted output variable 308. The accuracy of such predictions can vary based on the operation of gradient boosting trainer 302 and the quality of training data set 300. The goal is for gradient boosting model 304 to be as accurate as reasonably possible given a sufficiently rich training data set and a reasonable amount of time to spend on the training. This accuracy may be measured in various ways, as described in more detail below.

The operation of gradient boosting trainer 302 may involve training a set of decision trees (colloquially referred to as a “forest”), each of a limited depth or with a limited number of leaves. Thus, these trees are weak learners in that they generally do not take into consideration all available information in the training data set, and therefore their individual predictions may or may not have a high degree of accuracy. But gradient boosting makes overall predictions based on a weighting of the predictions from the individual trees. These overall predictions take into account most if not all of the training data set and therefore are likely to be more accurate than predictions from any of the individual trees.

But unlike a random forest, in which each tree is independent of the others, the construction of subsequent trees in a gradient boosting model can be based on the errors (or residuals) of one or more of the previously-constructed trees. In some cases, subsequent trees that compensate well for the errors of previous trees are given more weight toward the overall predictions, while in others all trees may be equally-weighted. Gradient boosting continues to construct trees in this fashion until it constructs a pre-determined number of trees or the new trees fail to improve the accuracy of the predictions by more than a pre-determined margin.

When the output variable takes on a continuous value, e.g., an integer falling with some range, trees are constructed based on the magnitude of residuals between actual values of the training data output variables and the associated predicted values. This may be referred to as gradient boosting for regression. In some cases, these residuals are called “pseudo-residuals” in order to differentiate gradient boosting from linear regression, but terms “residuals” and “pseudo-residuals” will be used interchangeably herein. The initial predictions for each observation i, p_{i,0}, may take on the same value p₀, such as the average of some or all output variables in the training data set. In other words, p₀=p_{i,0}∀ᵢ.

Here, the notation p_{i,0} refers to the prediction for observation i made by using trees 0 through n (see below for more details on how predictions are calculated using multiple trees). The initial prediction, p₀, may take the form of a single node rather than a tree, since it is commonly based only on values of the output variable.

In any event, the trees are constructed to predict the values of the residuals. The non-leaf nodes of the trees represent conditions of the input variables. For example, the root node in a tree constructed from training data set 300 might represent the condition X2>5 such that when this condition is true the node's left branch is followed, and when this condition is false the node's right branch is followed. Either of these branches might lead to another non-leaf node representing a condition or a leaf node representing a residual. More than two branches may be present, but binary trees are used in the examples herein for sake of convenience.

Tree construction may be based on various algorithms used for decision trees. In some cases, this may involve selecting an input variable and possibly an associated cutoff value that is based on entropy or Gini impurity. The cutoff value is selected so that it divides the values of the input variable in a fashion that makes the input variable reasonably predictive of the output variable. Then, the input variables are arranged as nodes in the tree with more predictive input variables generally being placed higher in the tree (e.g., closer to the root node). In some cases, randomness may be added to the process of determining where to place the input variables in the tree.

Since the number of observations is usually much greater than the number of leaves in a limited-size tree, the residuals of each observation that leads to the same leaf are typically averaged then placed in the leaf. Thus, a leaf can represent an aggregate residual, r_{i,0}, for a number of observations. Here, the notation r_{i,j} refers to the residual for observation i made in from using trees 0 through n (see below for more detail on how residuals are calculated using multiple trees).

Iterative predictions are then made for the observations in the training data set. Each prediction of the first iteration, p_{i,1}, involves traversing the tree 1 for an observation until reaching a leaf, and then adding that leaf's residual, r_{i,0}, to the initial prediction in accordance with a learning rate 0<α<1. In other words, p_{i,1}=p_{i,0}+αr_{i,0}. The learning rate helps prevent overfitting the training data set and allows small steps to be taken toward a higher prediction accuracy.

From the predictions p_{i,1}, new residuals r_{i,1} are calculated. Again, the residuals are based on differences between the actual output variable values in the training data set and the associated predicted values, with possible aggregation as described above. It is expected that these new residuals will generally be smaller than those of r_{i,0}, but this is not always the case for every residual.

The next tree, tree 2, may be constructed based on these new residuals. This tree may have the same structure as tree 1 or may be structured differently with nodes representing the input variables appearing in different locations (e.g., using randomness).

Then, each prediction of the second iteration, p_{i,2}, involves traversing both tree 1 and tree 2 for an observation until reaching their respective leaves, and then adding the associated residuals, r_{i,0} and r_{i,1}, to the initial prediction in accordance with the learning rate. In other words, p_{i,2}=p_{i,0}+αr_{i,0}+αr_{i,1}.

From the predictions p_{i,2}, new residuals r_{i,2} are calculated. It is expected that these residuals will continue getting smaller as the number of trees grows.

The process of constructing new trees based on residuals and making new predictions continues until, as noted above, a pre-determined number of trees are constructed or adding new trees fails to reduce the size of the residuals by more than a pre-determined margin. At this point, the training ends and the trained gradient boosting model is ready to make predictions for new observations of input variables.

Given such a new observation, the model begins with the initial prediction, p₀, and traverses all of the trees in accordance with the values of the input variables, adding the resulting residuals. Thus, assuming n trees in addition to the initial node, the predicted value of the output variable for a new observation is p_{new}=p₀+αΣⱼ₌₀ⁿrⱼ, where r₀ is the initial residual and rⱼ is for 1≤j≤n is the residual the jth tree for this observation.

Gradient boosting can also be used to predict the value of the output variable from a limited number of possible values. For example, when the output variable is Boolean, gradient boosting can be used to train a binary classifier. This may be referred to as gradient boosting for classification.

In this case, the predictions can be based on, across all observations in the training data set, (i) the natural log of the odds that the output variable is true, and (ii) the probability that the output variable is true. For instance, suppose that there are 100 observations with 70 being “true” and 30 being “false”. The natural logarithm of the odds that an observation is true would be ln(70/30)=0.847, while the probability that the output variable is true is 0.7.

The natural logarithm of the odds (0.847) is used as the initial prediction for all observations, and the probability (0.7) is used to calculate the residuals. Since 0.7 is greater than 0.5, the initial predictions are “true” for all observations (note that values other than 0.5 can be used as a cutoff in this process). Clearly, these initial predictions are not accurate, as indicated by their residuals. Assigning a value of 1.0 for true and 0.0 for false, the residuals will be 0.3 for each observation with an output variable that is “true” and −0.7 for each observation with an output variable that is “false”.

Further, the residuals are typically transformed to generate the output values of the leaves. This is because they are in terms of a probability while the predictions are in terms of the natural logarithm of the odds. An example transformation for a leaf with residuals rₖ as corresponding predicted probabilities ρₖ is:

$$\frac{{\sum }_{k} {r}_{k}}{{\sum }_{k} ( {ρ}_{k} ) ( 1 - {ρ}_{k} )}$$

Then these output values for the leaves are scaled by the learning rate and added to the initial predictions. Since these predictions are still in the form of the natural log of the odds, then can be converted to the probability form used by the residuals through application the logistic function. For instance, a predicted value of p would have a probability of:

$$\frac{{e}^{p}}{1 + {e}^{p}}$$

These residuals are then determined as the difference between the values of the output values from the training data set and the probabilities. As before, a new tree can be calculated based on these residuals. This process continues until a pre-determined number of trees have been constructed or adding new trees fails to reduce the size of the residuals by more than a pre-determined margin.

The trained gradient boosting model is then applied in a similar fashion to a new observation. The process adds the initial prediction and the transformed output values of each leaf associated with the new observation to find the predicted natural logarithm of the odds. Then, the logistic function is applied to this prediction to provide a probability. If the probability is greater than 0.5, the ultimate prediction for this new observation is “true”, otherwise it is “false”.

Note that the description above provides just a general overview of a few ways to carry out gradient boosting. Other techniques are possible. Further, other types of machine learning models, such as artificial neural networks or expert systems, can be used instead or in conjunction with gradient boosting techniques. Nonetheless, gradient boosting generally works well with certain types of data and produces models that are explainable—one can analyze the model to understand how and why it is producing its answers, which might not be the case for artificial neural networks. Moreover, gradient boosting models also tend to perform well when the training data set is sparse (e.g., many observations with at least one missing input variable), whereas artificial neural networks tend to be less efficient with sparse data.

There are two popular gradient boosting frameworks that can be used to train and deploy gradient boosting models, XGBoost and LightGBM. Each is briefly discussed below for purposes of example. Nonetheless, other frameworks can be used.

### A. XGBoost

When the output variable takes on a continuous value, XGBoost builds trees by placing all residuals in a root node, and then calculates a similarity score for the residuals. The similarity score can be calculated as the square of the sum of the residuals divided by the number of residuals. In some cases, a regularization constant is also added to the denominator to reduce sensitivity to outliers and overfitting of the training data set. Regardless, the higher the similarity score, the more similar the residuals.

Then various ways of dividing the residuals into groups are considered, to see if any of these divisions results in a higher overall similarity score. This can be determined by calculating a gain for a division over the original grouping of all residuals in the root node. The gain can be the similarity score of the root node subtracted from the sum of similarity scores for each node of the divisions. Then, the division that produces the largest gain of all divisions is selected as to produce branches from the root node (i.e., each group of residuals in the selected division become a child node of the root node).

Then, the same process is performed for each of the new child nodes. If a child node contains only one residual, it cannot be further divided and becomes a leaf. Also, trees may be limited to a maximum number of levels (e.g., 4, 6, or 8) and a node at this maximum depth would also not be further divided. In some cases, XGBoost may require that a minimum number of residuals (e.g., 2, 3, 4 . . . ) be represented in each node.

Once an XGBoost tree is constructed in this fashion, some branches that produce less than a threshold gain may be pruned. The pruning process recombines them into their respective parent nodes.

Predictions are made by traversing the tree with the values of the input variable until a leaf is reached. The output value of the leaf is the sum of the residuals in that leaf divided by the number of residuals in that leaf. Again, the regularization constant may also be added to the denominator.

Like standard gradient boosting, this tree is then used to make predictions that are scaled by a learning rate. The residuals from these predictions are then used to construct the next tree, and so on. Tree construction ends when a pre-determined maximum number of trees have been constructed or the residuals become smaller than a pre-determined threshold.

When the output variable takes on a one of a discrete number of values (e.g., for classification), XGBoost maps these into a numeric range. For example, each value for a Boolean output variable would be mapped to 1.0 or 0.0. Then, the divisions are made as described above.

However, a different similar score calculation per node is used, this one being the square of the sum of residuals in the node divided by the sum over all observations the product of (i) the previous probability and (ii) the previous probability subtracted from one. The regularization constant may also be added to the denominator. Tree construction also occurs as described above, though using this different similarity score calculation to determine gain.

As noted, XGBoost may require that a minimum number of residuals be represented in each leaf. In this version of XGBoost, however, a value called “cover” is used instead of a count of residuals. Cover is the denominator of the similarity score minus the regularization constant. Leaves with less than a threshold value of cover may be removed from the tree, effectively pruning the tree. The other pruning techniques described above may also be used.

For prediction, the output value of a leaf is the sum of residuals in the leaf divided by the sum over all observations the product of (i) the previous probability and (ii) the previous probability subtracted from one. The regularization constant may also be added to the denominator. Across multiple trees, the prediction for an observation is the natural logarithm of the odds for the initial output value, added to the output values for each tree scaled by the learning rate.

The logistic function can be applied to this result in order to convert it back into a probability. Based on the value of this probability (e.g., above or below 0.5 for a binary output variable), a value of the output variable can be selected.

XGBoost also employs a number of techniques that speed up its processing for large training data sets. These techniques include using an approximate greedy algorithm for selecting divisions, weighted sketch algorithms for focusing on observations that are hard to predict, distributed training across multiple processors or computers, and/or keeping commonly-used variables and constants in the processor cache. Other techniques can also be applied.

### B. LightGBM

LightGBM also employs gradient boosting but does so in a way that generally increases training speed, reduces memory utilization, and provides improved accuracy. Particularly, rather than consider all values of an input variable, LightGBM bins these values to form a histogram, and operates on the bins rather than the values. Also, LightGBM uses exclusive feature bundling to reduce dimensions of the feature space when two or more features tend to take on mutually exclusive values. Further, LightGBM uses gradient-based one side sampling to identify the observations with the largest residuals and operate only on the observations and well as a random sampling of observations with lower residuals.

As a result, LightGBM focuses computation where it is needed most—on input variables that are dissimilar from one another and observations for which early trees have the most error. Thus, in practice, LightGBM can perform about ten times faster than other gradient boosting implementations with similar accuracy.

### III. SURVIVAL TIME ANALYSIS

Survival time analysis is a set of statistical methods that provide estimates of the amount of time (e.g., days, weeks, month, years) until an event occurs. An example use of such an analysis would be to predict the number of days between when a patient is diagnosed with a condition (e.g., portal hypertension) and when the patient is expected to exhibit an outcome, such as an indication or diagnosis of a further condition (e.g., varices, ascites, or death). This analysis can occur for both patients who are being treated as well as for patients who are untreated. Often, a goal of survival time analysis is to determine whether a specific treatment has an impact on the predicted survival time.

Notably, survival time analysis is not limited to predicting the time until a patient dies—it is used to predict the time between two events. However, it is often used to predict patient survival times, so thus the name.

Survival time analysis is typically implemented as a form of regression, with a training data set of observations. Each observation may indicate whether a patient exhibited an outcome (a binary value of true or false) and if so, the time between some initial state and that outcome. For example, the initial state may be a diagnosis of portal hypertension and the outcome may be varices. If the patient was diagnosed with varices within the observation period, the amount of time between the two diagnoses is the survival time. Since some conditions (like portal hypertension) have more than one possible progression, each may be modeled separately.

Most survival time analyses must account for censored data. For instance, in a clinical trial some patients may drop out of the trial before an outcome can be observed for them. This may be due to various reasons, such as the study ending before the outcome is observed in the patient, or patient leaving the study early for some reason (e.g., loss of interest, moving to a different location, or death). Such data is considered to be “right censored” and is typically assumed to be non-informative. In order words, right censored observations are not taken into account by the model.

Cox regression is a survival time analysis technical that allows multiple input variables to be used to predict an outcome (the output variable). The Cox model assumes that the log-hazard of an observation is a linear function of its covariates and a population-level baseline hazard function that varies over time. Since the embodiments herein involve training data sets with multiple input variables, and the influence of each on various outcomes is unknown, Cox regression is a good candidate for predicting survival times.

Another method for survival time analysis is accelerated failure time (AFT). AFT is also a regression-based approach that supports multiple input variables. Unlike Cox regression, AFT allows a fully parametric specification of the hazard function. Nonetheless, other parametric, semi-parametric, or non-parametric survival functions can be used.

XGBoost implementations support Cox regression and AFT, and thus can be used for survival analysis. Other gradient boosting implementations may provide similar functionality. Another software package, NGBoost, includes AFT features integrated into a gradient boosting framework (e.g., as random survival forests) that allows gradients to be characterized as probability distributions.

### IV. PREDICTING PROGRESSION OF PORTAL HYPERTENSION

As noted, portal hypertension is a condition in which damage to the liver (e.g., cirrhosis), or obstructions of the portal vein (intrinsic or extrinsic), leads to elevated venous blood pressure in the portal venous system, which carries blood from gastrointestinal organs to the liver. Untreated, portal hypertension can lead to a number of conditions, including but not limited to varices, variceal hemorrhages, recurrent variceal hemorrhages, ascites, refractory ascites, hepatic encephalopathy, recurrent hepatic encephalopathy, jaundice, lower extremity swelling, coagulopathy, pulmonary complications, portosystemic shunts, and death.

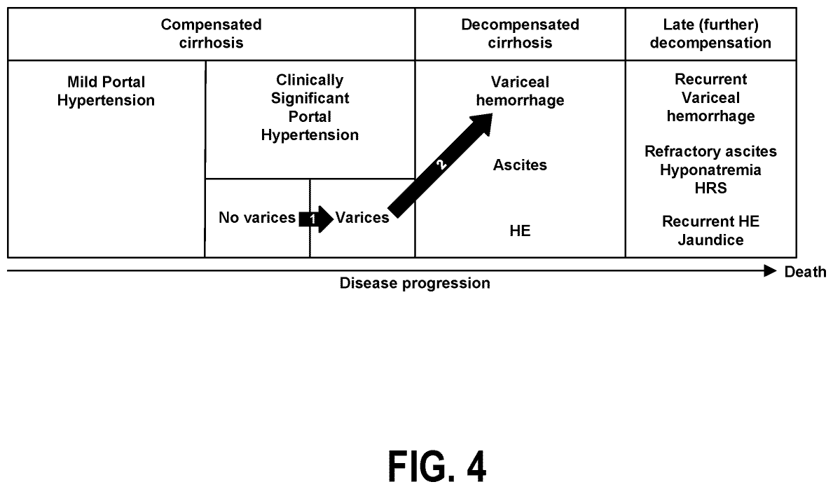

FIG. 4 depicts example progression paths for portal hypertension. These paths general progress from observations of compensated cirrhosis to observations of decompensated cirrhosis to observations of further decompensation.

A patient with compensated cirrhosis may be largely asymptomatic and might not have ascites, variceal hemorrhages, hepatic encephalopathy, or jaundice. Nonetheless, such a patient could be diagnosed with mild portal hypertension (usually with no observed conditions). Mild portal hypertension may progress to clinically significant portal hypertension (in which one of more conditions have been observed, such as varices).

Clinically significant portal hypertension may progress to decompensated cirrhosis. A patient with decompensated cirrhosis may exhibit one or more conditions (e.g., ascites, variceal hemorrhages, hepatic encephalopathy, and/or jaundice). A patient with late-stage decompensated cirrhosis may have more severe conditions, such as recurrent variceal hemorrhages, refractory ascites, hepatic encephalopathy, portosystemic shunts, and/or jaundice. The life expectancy for patients with decompensated cirrhosis may be counted in months or a small number of years.

Thus, a possible progression path for portal hypertension is from compensated cirrhosis with no varices to compensated cirrhosis with varices, to decompensated cirrhosis with variceal hemorrhages, to recurrent variceal hemorrhages, to death. Other progression paths are possible.

Current treatments for portal hypertension either lack effectiveness for many patients or are invasive. Thus, it is desirable to have a framework for evaluating whether a particular patient is likely to progress to one or more of these conditions, as well as a prediction of the time frame of progression. With such a framework at hand, patients who are likely to progress faster toward an undesirable condition can be identified early in their progression. These patients can then be considered for more aggressive treatment in order to slow their progression. In other situations, patients with any predicted progression speed may be selected for clinical trials of new treatments (e.g., diet, supplements, and/or pharmaceuticals). The embodiments herein may include software that analyzes a database of patients, for example, and provides a ranking of these patients for inclusion in a clinical trial in order of risk of progression (trials with patients having higher risks of progression can shorten the length of the trial and reduce the placebo effect). Thus, the embodiments herein can potentially lead to improved lifespans and improved quality of life for portal hypertension patients.

Thus, an array of n machine learning models—one for each condition of interest—may be trained on patient data to predict whether a condition is expected to occur, and the length of time until the condition is expected to be observable. For example, there may be one model for each of varices, ascites, variceal hemorrhages, hepatic encephalopathy, and death. Therefore n=5 in this example. A further overall model may be trained on the patient data to predict whether any of these conditions is expected to occur, and the length of time until that condition is expected to be observable. Thus, there may be a total of n+1 machine learning models. In some cases, models may predict progressions of earlier complications to later, more severe complications (e.g., from portal hypertension with varices without bleeding to varices with bleeding).

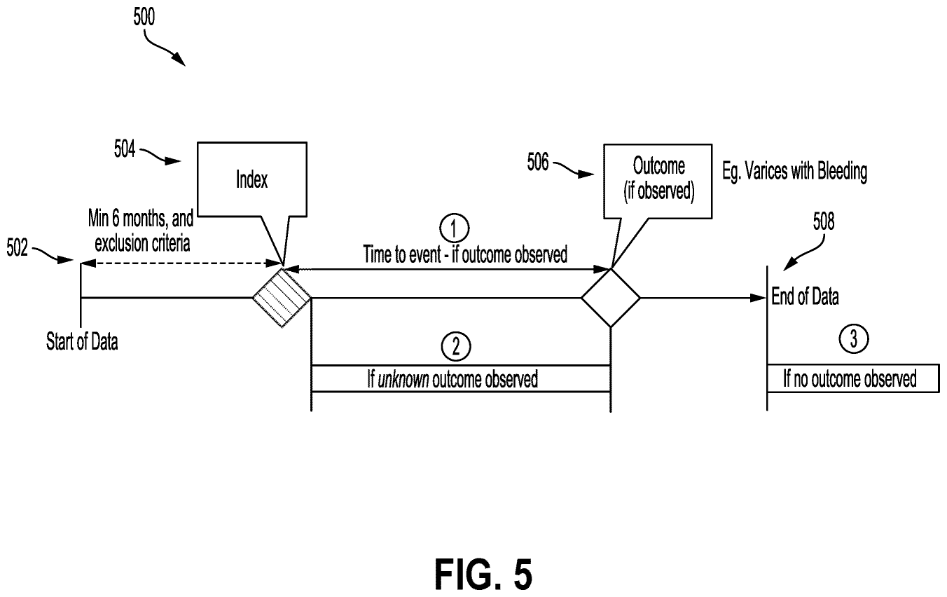

These machine-learning models may operate based on observed progression timelines. A general framework for such a timeline is illustrated in FIG. 5. Particularly, timeline 500 includes four main points of interest for modeling possible progressions. These progressions include where an outcome is observed, an unknown outcome is observed, or no outcome is observed. Each patient's portal hypertension progression can be fit into timeline 500.

Point 504 is the index date, which is a reference date for the prediction. The index date may be the first recorded diagnosis of portal hypertension and/or cirrhosis for a patient, or may be the point from which a prediction is made for a patient already diagnosed with portal hypertension and/or cirrhosis. In some cases, these patients are selected so that they have no prior recorded complication of portal hypertension or cause of non-cirrhotic portal hypertension. Patients exhibiting certain characteristics or not observing certain characteristics may be excluded from the training data, such as patients with a liver transplant or severe cirrhosis prior to this index date.

Thus, point 504 is from when the time to an outcome (if any) is measured. In practice, it is desirable for there to be at least 6 months of observations prior to point 504, increasing the chance of identifying incident patients. Thus, the time between point 502 and point 504 should be at least 6 months, though minimum time periods other than 6 months may be used.

Point 506 is the time at which an outcome is observed. As noted, in some cases no outcome is observed. In those situations, point 506 will not exist. Outcomes may be known (e.g., varices, variceal hemorrhages, recurrent variceal hemorrhages, ascites, refractory ascites, hepatic encephalopathy, recurrent hepatic encephalopathy, and/or jaundice). In other situations, outcomes may be unknown. For example, a condition may have been observed but the exact nature of the condition cannot be determined from the data available (e.g., it may be known that a patient has varices but whether the varices are bleeding might be unknown because the patient has yet to have an endoscopy to make that determination).

Point 508 represents an end of the data for a patient for which no outcome was observed. For instance, the patient may have died, dropped out of the healthcare system that was collecting the data, or the study period ended. Such a patient's data may be right censored.

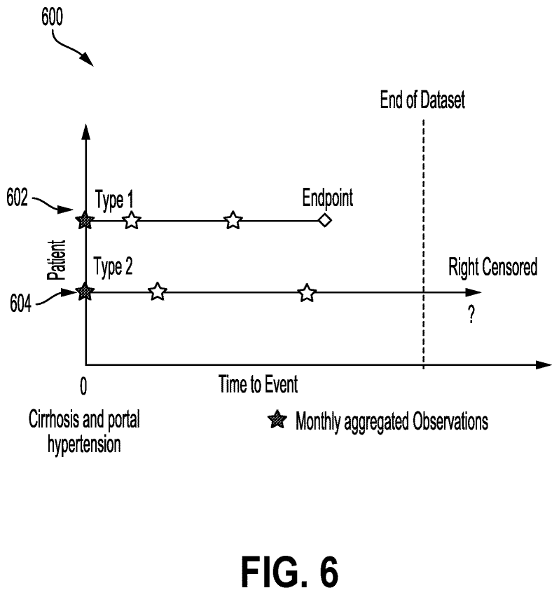

FIG. 6 depicts this patient data in another way. Graph 600 includes two example timelines for progression of portal hypertension. Both assume that patients have been diagnosed with cirrhosis and portal hypertension. Both also include monthly aggregated health data (e.g., results of examinations, procedures, vital sign measurements, and/or lab tests), represented by stars. For sake of simplicity, only three stars are shown per timeline in FIG. 6, but these health data entries may continue for many months. Regardless, the health data may be considered a sparse data set, with some months missing at least some values. It is possible that up to 20%-60% of all expected health data entries might not be present.

Timeline 602 is referred to as a “type 1” timeline, as it shows progression of liver disease to an outcome (e.g., varices, variceal hemorrhages, recurrent variceal hemorrhages, ascites, refractory ascites, hepatic encephalopathy, recurrent hepatic encephalopathy, and/or jaundice). Timeline 604 is referred to as a “type 2” timeline, as there is no observed progression of liver disease and the associated data is right censored.

Patient health records may be formed into a training data set in accordance with such timelines. As a possible example, an index date is determined for each patient. This could be the date at which a patient's data in considered for inclusion in the training data set. Any patient for whom portal hypertension has been observed for at least 6 months prior to the index date may have their data incorporated into the training data set. Then, for patients with known or unknown outcomes, respective outcomes and associated outcome times are determined. Patients with no outcomes are right censored. Alternatively, progressions may be represented as an amount of time (e.g., in days) between points 504 and 506 with an indication of the outcome observed at point 506.

Some data may also be left censored, as certain unknown outcomes can define the lower limit of progression time to a condition. For example, if the patient did not exhibit varices prior to a give date, does exhibit varices now, and the patient's bleeding status is unknown.

TABLE 1

| Patient | Index date | Outcome | Time to Outcome (days) |
|---|---|---|---|
| A | May 22, 2019 | Varices | 432 |
| B | Jun. 1, 2019 | Refractory Ascites | 325 |
| C | May 27, 2019 | Unknown | 517 |

Table 1 provides a simple example of possible training data. In this table, patient A was observed to have varices after 432 days from an index date of May 22, 2019, patient B was observed to have refractory ascites after 325 days from an index date of Jun. 1, 2019, and patient C was observed to have an unknown condition after 517 days from an index date of May 27, 2019. In some cases, multiple entries per patient may be possible when multiple conditions were observed. For example, there could be a second entry for patient A in Table 1 that indicates that patient A was also observed to have varices with bleeding after 489 days. Alternatively or additionally, there may also be entries for the monthly aggregated health data (not shown). Monthly lab results may be aggregated over the prior 6 months leading up to the index date or observation, with the last observed value carried forward until the index date.

Regardless, the format and content of Table 1 is for purposes of illustration, and other formats and/or content of training data may be possible. As noted, the training data may also include, for each patient, demographic data, comorbidities, lab results, vital signs, and/or other health data (e.g., medications, prescriptions, treatments, exercise, nutrition, mental wellbeing), etc.

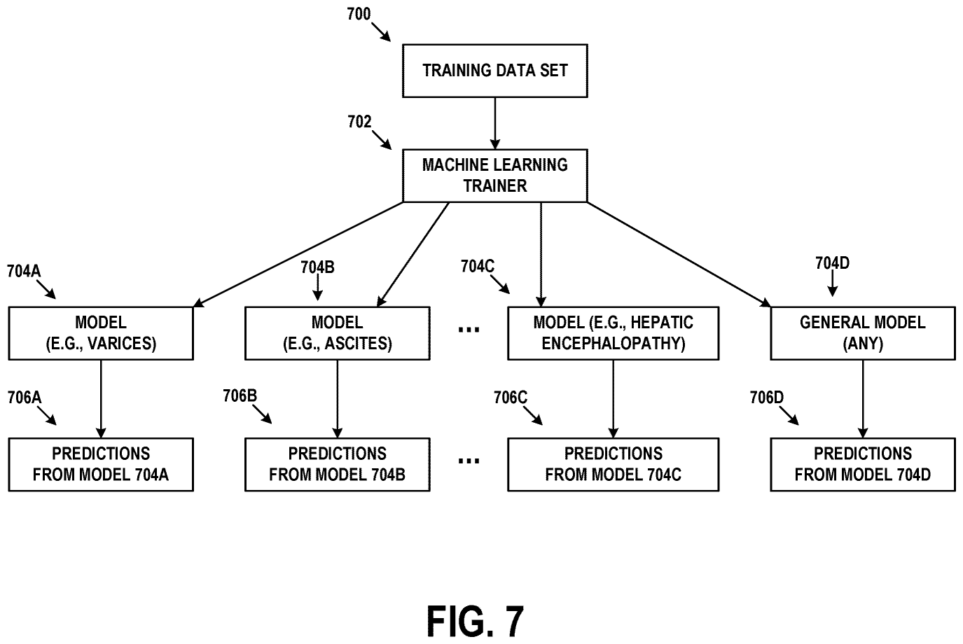

FIG. 7 depicts the training and prediction phases of an array of machine learning models. Training data set 700 may include observations from patients that relate to progression of portal hypertension. As noted, these may comprise input variables (e.g., demographics, comorbidities, lab results, and/or vital signs) and output variables (indications of diagnoses of one or more further conditions and the number of days from an index date to when one or more of these further conditions was diagnosed).

Machine learning trainer 702 may be applied to training data set 700 to produce an array of n trained machine learning models. Machine learning trainer 702 may employ gradient boosting and survival time aspects in a manner that is robust when training data set 700 is sparse.

Each trained model may predict, given new input variables from a patient (not shown), whether that patient is expected to be diagnosed with a specific condition and how many days are expected before such a diagnosis can be made. For example, model 704A may be trained to make predictions 706A of whether a diagnosis of varices will be made and if so how many days from the index date to the diagnosis. Likewise, model 704B may be trained to make predictions 706B of whether a diagnosis of ascites will be made and if so how many days from the index date to the diagnosis. Similarly, model 704C may be trained to make predictions 706C of whether a diagnosis of hepatic encephalopathy will be made and if so how many days from the index date to the diagnosis. Each of these models may be independently trained and may also operate independently when making predictions.

The predictions of whether a patient is expected to be diagnosed with a specific condition may be in the form of a hazard ratio of whether the individual is expected to exhibit progression to a condition related to portal hypertension or cirrhosis. The hazard ratio may be in reference to the general population, i.e., whether the patient is expected to progress faster or slower. This hazard ratio could be a non-negative value used to compare a specific cohort to a general population. Thus, a hazard ratio of 0.1 means that the specific cohort is 10 times less likely to exhibit a condition and a hazard ratio of 2 means that the specific cohort is twice as likely to exhibit the condition. A hazard ratio may be thresholded into a Boolean true or false value for inclusion or exclusion (e.g., if the hazard ratio is above 0.1 the Boolean value is “true”, and if the hazard ration is at or below 0.1, the Boolean value is “false”).

Or, put in the context of FIG. 3, the output variable in training data set 300 may be a time range, where the upper and lower bounds of this range are the same if the event was observed at an exact time. Further, predicted output variable 308 may be a hazard ratio and/or a predicted time value (e.g., a number of days).

Additionally, machine learning trainer 702 may be applied to training data set 700 to further produce a general model (making a total of n+1 trained models) that predicts whether any such condition will be diagnosed and how many days are expected before such a generic diagnosis can be made. For patients who are predicted to be diagnosed with more than one condition (e.g., varices and ascites), the predicted number of days may be from the index date to when the earliest diagnosis is expected to occur. Thus, model 704D may be trained to make predictions 706D of whether diagnosis of any one or more conditions related to varices will be made and if so how many days from the index date to the diagnosis.

As shown by the ellipses in FIG. 7, n may take on various values. In some cases, n could be 1 or 2, and in others n could be 7, 8, or even higher. Thus, the embodiments herein may use any value of n. These embodiments might or might not include a general model.

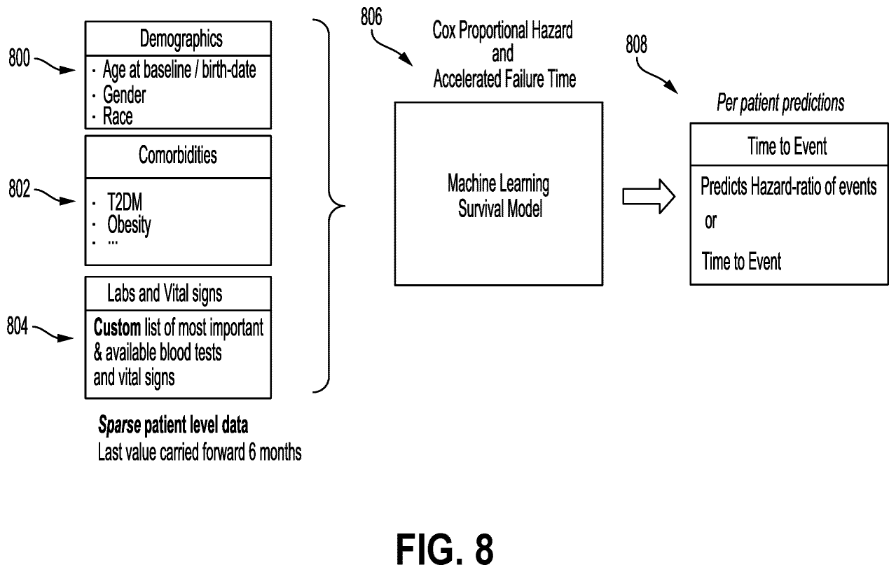

FIG. 8 depicts how any one of these trained models make predictions. Demographic data 800 (e.g., age, gender, race), comorbidities 802 (e.g., diabetes, obesity), and lab results and vital sign data 804 (e.g., body mass index, blood pressure, heart rate, blood test data) are provided as input to trained machine learning model 806. Other data, such as medications taken, may be included in the input.

As noted, the training data set may indicate an index date for a patient representing a point in time at which the patient is known to have or is at least suspected to have portal hypertension and/or cirrhosis. Further, lab results and vital sign data 804 may include multiple measurements made over time, optionally aggregated on a monthly basis.

Machine learning model 806 may employ some form of survival time technique (e.g., Cox proportional hazard and/or accelerated failure time) to predict the number of days from the index date until a further condition is diagnosed. Machine learning model 806 may be based on LightGBM or XGBoost as shown, or it may be based on NGBoost or some other gradient boosting technique that supports survival analysis. In alternative embodiments, machine learning model 806 may be based at least in part on an artificial neural network, expert system, or some ensemble combination of any of these or other models.

Machine learning model 806 may produce per-patient predictions 508, which may indicate whether a condition is expected to be observed and the number of data between the index date and when the condition is expected to be observed.

Data for training and validating machine learning model 806 may come from a variety of sources, including hospitals, health care providers, clinical sources, and/or insurance claims. For example, machine learning model 806 could be trained on insurance claim data, as that data may include demographics, vital signs, and blood test results for patients with and without portal hypertension and/or cirrhosis. This training data may be pre-processed in various ways, e.g., to remove outliers, de-skew, and/or normalize.

Machine learning model 806 as trained can then be validated on clinical data to determine to what extent it accurately predicts the progression of portal hypertension in patients. Once validated, machine learning model 806 can be applied to identify patients in hospitals or using hospital services that are candidates for further testing, treatment, or inclusion in clinical trials.

In some cases, the training data may be gathered from multiple geographic regions. However, the data may be segmented per region to develop region-specific models. In some situations, information from identified patients may be checked for novelty—e.g., whether the input variables for these patients are consistent with those in the training data. To do so, a similarity model might be applied to the information from identified patients as well as the training data, with dissimilar patients being identified for further processing before a prediction is finalized. This can be helpful if the machine learning model is trained on data from one population of individuals (e.g., located in North America) but applied to another population of individuals (e.g., located in Europe).

### V. EXPERIMENTAL RESULTS

The embodiments herein were applied to training data including a cohort of 10,429 patients that matched the inclusion and exclusion criteria, with 41% of these patients having a severe condition observed. The median progression time for these patients was 190 days. Patient data was collected from the Optum Clinformatics Data Mart (Optum) data set (de-identified US electronic health records, 2007-2021). Hazard rates for progression were modeled using a machine learning survival-time model robust to sparse data. Model performance was evaluated using 3-fold cross validation of the cumulative dynamic area-under-the-curve (AUC) and compared with four established liver disease scores (fib-4, ALBI, PALBI, and MELD), as well as patient age as a baseline.

The AUC metric can be based on receiver operating characteristic (ROC) curves or a precision-recall curve generated from model results. ROC curves plot the true positive rate (number of true positives divided by the sum of true positives and false negatives) versus the false positive rate (number of false positives divided by the sum false positives and true negatives) of the model. An ROC curve visually describes tradeoffs between the true positive rate and the false positive rate. One measure of model quality is the AUC for the ROC curve. This value is typically between 0.5 and 1.0, with higher values being indicative of better model performance across various parameter settings. Precision-recall curves plot the precision (the number of true positives divided by the sum of the true positives and the false positives) versus the recall (the number of true positives divided by the sum of true positives and false negatives) of the model. Again, AUC can be used to evaluate model quality, with higher values indicating more quality.

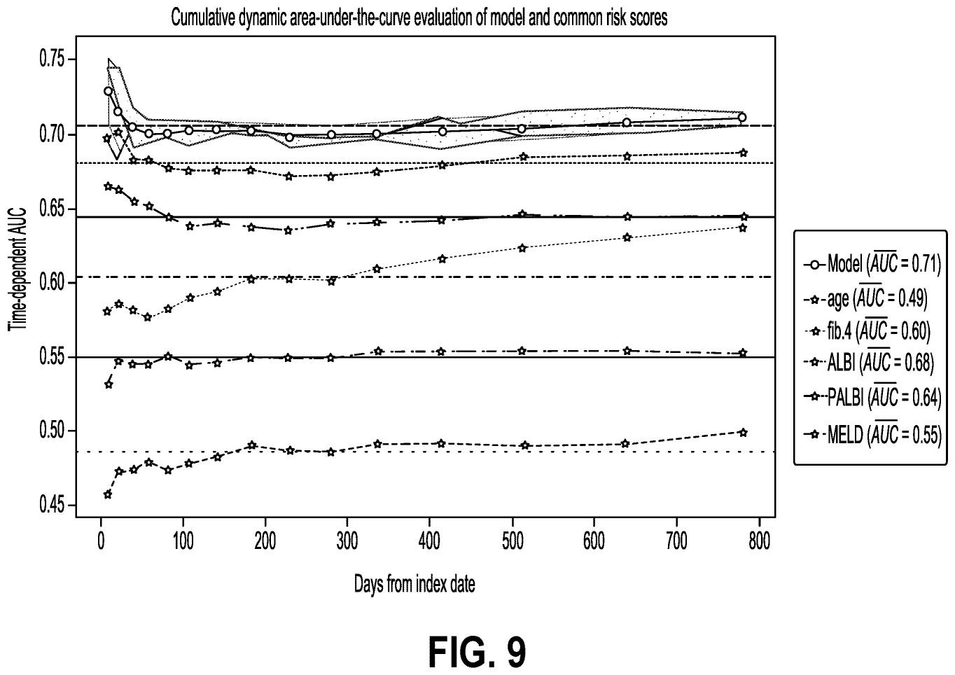

FIG. 9 depicts an evaluation of the models in a graph, using the general model that predicts progression to any of the conditions. As shown, the embodiments herein produce a model that outperforms all of the fib-4, ALBI, PALBI, and MELD techniques, at least in terms of its ability to predict disease progression for patients with portal hypertension. As a baseline for comparison, age was also used as a predictor, which resulted in an AUC Of 0.49, no better than a random guess.

Thus, the prediction model described herein offers an improvement in AUC versus conventional techniques for predicting progression of portal hypertension. The model also performs well on partially observed data and the condition-specific models can differentiate risk of progression by individual complications, which is an improvement to existing techniques.

### VI. DEPLOYMENT SCENARIOS

The embodiments herein may be deployed in a number of arrangements. With regard to the training of one or more machine learning models, this may take place on one or more computing devices within a server cluster, such as in a public cloud network (e.g., Amazon AWS or Microsoft Azure) or on a private system. With regard to the execution of these models on new observations, the models could be hosted in various locations and environments.

In one possible example, the trained models may be hosted on a public cloud network or on a private network, and provide results to client devices either via a web or application interface. For instance, the client device may transmit a request to a remotely hosted model, the request containing a set of new input variables comprising the new observation. The model may take these as input and produce a corresponding output that is then transmitted to the client device in response to the request. These trained models may be operated by various entities, such as a hospital, hospital network, physician, physician network, university, pharmaceutical company, or some consortia of one or more of these to other entities.

Alternatively, the trained model may be packaged with a client application that can be downloaded and installed on a desktop, laptop, or mobile computing device. Thus, the client application would contain a user interface that allows a user to enter or otherwise indicate the input variables for a new observation. The client application would then apply the model this new observation and produce a corresponding output that is displayed and/or stored by the client device. This scenario has the advantage that a live network connection is not required to use the model.

In other alternatives, the trained model may be used to develop simple clinical prediction rules, such as a decision tree, that a health care provider can follow to predict portal hypertension progression and/or prognoses.

Other deployment scenarios may exist. Thus, the embodiments herein are not limited to these scenarios.

### VII. EXAMPLE OPERATIONS

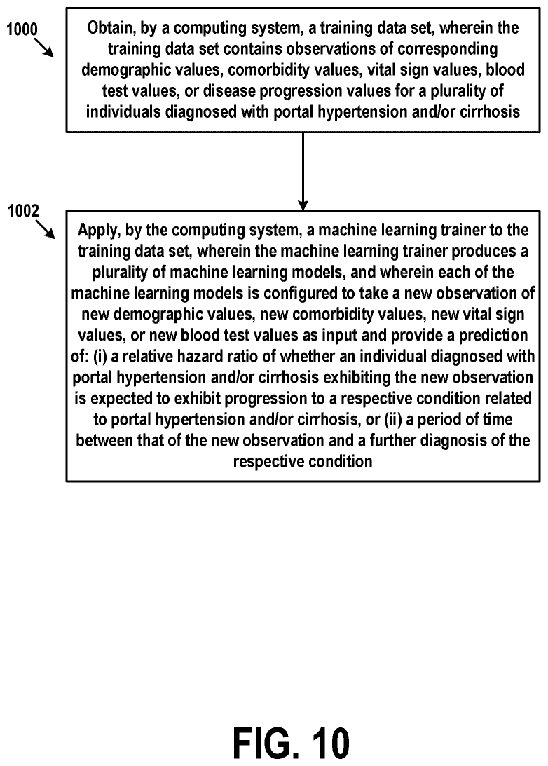

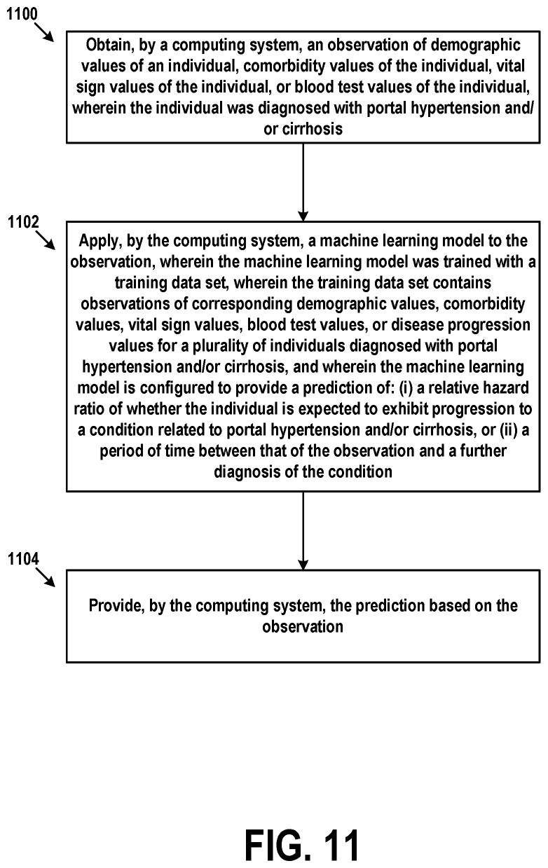

FIGS. 10 and 11 are flow charts illustrating example embodiments. The operations illustrated by FIGS. 10 and 11 may be carried out by a computing system or computing device that includes a software application configured to perform any of the embodiments herein. Non-limiting examples of the computing system or computing device include computing device 100 or server cluster 200, for example. However, the operations can be carried out by other types of devices or device subsystems. For example, the operations could be carried out by a portable computer, such as a laptop or a tablet device.

The embodiments of FIGS. 10 and 11 may be simplified by the removal of any one or more of the features shown therein. Further, these embodiments may be combined with features, aspects, and/or implementations of any of the previous figures or otherwise described herein. Such embodiments may include instructions executable by one or more processors of the one or more computing devices of the system or virtual machine or container. For example, the instructions may take the form of software and/or hardware and/or firmware instructions. In an example embodiment, the instructions may be stored on a non-transitory computer readable medium. When executed by one or more processors of the one or more computing devices, the instructions may cause the one or more computing devices to carry out various operations of the embodiments.

In these embodiments, an individual diagnosed with portal hypertension and/or cirrhosis may include the individual being diagnosed with compensated cirrhosis, the individual having compensated cirrhosis, or the individual exhibiting symptoms of compensated cirrhosis. Thus, the term “portal hypertension and/or cirrhosis” may include compensated cirrhosis without a specific diagnosis of portal hypertension.

Block 1000 of FIG. 10 involves obtaining, by a computing system, a training data set, wherein the training data set contains observations of corresponding demographic values, comorbidity values, vital sign values, blood test values, and/or disease progression values for a plurality of individuals diagnosed with portal hypertension and/or cirrhosis.

Block 1002 of FIG. 10 involve applying, by the computing system, a machine learning trainer to the training data set, wherein the machine learning trainer produces a plurality of machine learning models, and wherein each of the machine learning models is configured to take a new observation of new demographic values, new comorbidity values, new vital sign values, and/or new blood test values as input and provide a prediction of: (i) a hazard ratio of whether an individual diagnosed with portal hypertension and/or cirrhosis exhibiting the new observation is expected to exhibit progression to a respective condition related to portal hypertension or cirrhosis, and/or (ii) a period of time between that of the new observation and a further diagnosis of the respective condition.

In some embodiments, the machine learning trainer also produces a further machine learning model configured to take the new observation as input and provide a further prediction of: (i) a further hazard ratio of whether the individual is expected to exhibit progression to any condition related to portal hypertension or cirrhosis, and/or (ii) a further period of time between that of the new observation and an additional diagnosis of any condition related to portal hypertension or cirrhosis.

In some embodiments, the disease progression values for a particular individual of the plurality of individuals includes an index date and one or more outcomes, and wherein each of the one or more outcomes indicates a particular condition and an observed period of time between its index date and when the particular condition was diagnosed.

In some embodiments, the disease progression values also include one or more additional outcomes, wherein each of the one or more additional outcomes indicates an unknown condition and an additional observed period of time between the index date and when the unknown condition was identified. For example, in a patient with varices, the bleeding status of the condition might be unknown.

In some embodiments, there is at least six months of vital sign values or blood test values prior to the index date in the disease progression values for the plurality of individuals.

In some embodiments, the particular condition is one of varices, variceal hemorrhages, recurrent variceal hemorrhages, ascites, refractory ascites, hepatic encephalopathy, recurrent hepatic encephalopathy, portosystemic shunts, or jaundice.

In some embodiments, the demographic values include ages, genders, races, or ethnicities of the plurality of individuals.

In some embodiments, the vital sign values include body mass indices, blood pressure readings, or heart rates of the plurality of individuals.

In some embodiments, the comorbidity values include indications of diabetes or obesity.

In some embodiments, values within the training data set are 20%-60% populated.

In some embodiments, the machine learning models are based on gradient boosting.

In some embodiments, the machine learning models are based on gradient boosting and survival time analysis.

In some embodiments, the training data set includes at least 10,000 observations gathered from medical claim records or electronic health records.

In some embodiments, the hazard ratio is provided as a Boolean indication of progression to the respective condition (e.g., the hazard ratio may be thresholded to either a value of “true” indicating that progression to the respective condition is predicted, or “false” indicating that no progression to the respective condition is predicted).

In some embodiments, the observations in the training data set also include indications of medications, prescriptions, or treatments relating to the plurality of individuals, wherein the new observation also includes indications of medications, prescriptions, and/or treatments relating to the individual.

Block 1100 of FIG. 11 involves obtaining, by a computing system, an observation of demographic values of an individual, comorbidity values of the individual, vital sign values of the individual, and/or blood test values of the individual, wherein the individual was diagnosed with portal hypertension and/or cirrhosis.

Block 1102 of FIG. 11 involves applying, by the computing system, a machine learning model to the observation, wherein the machine learning model was trained with a training data set, wherein the training data set contains observations of corresponding demographic values, comorbidity values, vital sign values, blood test values, and/or disease progression values for a plurality of individuals diagnosed with portal hypertension and/or cirrhosis, and wherein the machine learning model is configured to provide a prediction of: (i) a hazard ratio of whether the individual is expected to exhibit progression to a condition related to portal hypertension or cirrhosis, and/or (ii) a period of time between that of the observation and a further diagnosis of the condition.

Block 1104 of FIG. 11 involves providing, by the computing system, the prediction based on the observation.

Some embodiments may further involve applying, by the computing system, a second machine learning model to the observation, wherein the second machine learning model was trained with at least part of the training data set, and wherein the second machine learning model is configured to provide a second prediction of: (i) a second hazard ratio of whether the individual is expected to exhibit progression to a second condition related to portal hypertension or cirrhosis, and/or (ii) a second period of time between that of the observation and a second further diagnosis of the second condition; and providing, by the computing system, the second prediction based on the observation.

Some embodiments may further involve applying, by the computing system, a further machine learning model to the observation, wherein the further machine learning model was trained with at least part of the training data set, and wherein the further machine learning model is configured to provide a further prediction of: (i) a further hazard ratio of whether the individual is expected to exhibit progression to any condition related to portal hypertension or cirrhosis, and/or (ii) a further period of time between that of the observation and a further diagnosis of any condition related to portal hypertension or cirrhosis; and providing, by the computing system, the further prediction based on the observation.

In some embodiments, providing the prediction comprises displaying the prediction on a graphical user interface.

In some embodiments, obtaining the observation comprises receiving the observation from a client device in communication with the computing system over a network, and wherein providing the prediction comprises transmitting the prediction to the client device.

In some embodiments, the disease progression values for a particular individual of the plurality of individuals includes an index date and one or more outcomes, wherein each of the one or more outcomes indicates a particular condition and an observed period of time between its index date and when the particular condition was diagnosed.

In some embodiments, the disease progression values also include one or more additional outcomes, wherein each of the one or more additional outcomes indicates an unknown condition and an additional observed period of time between the index date and when the unknown condition was identified.

In some embodiments, there is at least six months of vital sign values or blood test values prior to the index date in the disease progression values for the plurality of individuals.

In some embodiments, the particular condition is one of varices, variceal hemorrhages, recurrent variceal hemorrhages, ascites, refractory ascites, hepatic encephalopathy, recurrent hepatic encephalopathy, portosystemic shunts, or jaundice.

In some embodiments, the demographic values include ages, genders, races, or ethnicities of the plurality of individuals.

In some embodiments, the vital sign values include body mass indices, blood pressure readings, or heart rates of the plurality of individuals.

In some embodiments, the comorbidity values include indications of diabetes or obesity.

In some embodiments, values within the training data set are 20%-60% populated.

In some embodiments, the machine learning model is based on gradient boosting.

In some embodiments, the machine learning model is based on gradient boosting and survival time analysis.

In some embodiments, the training data set includes at least 10,000 observations gathered from medical claim records or electronic health records.

In some embodiments, the hazard ratio is provided as a Boolean indication of progression to the respective condition (e.g., the hazard ratio may be thresholded to either a value of “true” indicating that progression to the respective condition is predicted, or “false” indicating that no progression to the respective condition is predicted).

In some embodiments, the observations in the training data set also include indications of medications, prescriptions, or treatments relating to the plurality of individuals, wherein the observation also includes indications of medications, prescriptions, or treatments relating to the individual.

### VIII. CLOSING

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid-state drive, or another storage medium.

The computer readable medium can also include non-transitory computer readable media such as non-transitory computer readable media that store data for short periods of time like register memory and processor cache. The non-transitory computer readable media can further include non-transitory computer readable media that store program code and/or data for longer periods of time. Thus, the non-transitory computer readable media may include secondary or persistent long-term storage, like ROM, optical or magnetic disks, solid-state drives, or compact disc read only memory (CD-ROM), for example. The non-transitory computer readable media can also be any other volatile or non-volatile storage systems. A non-transitory computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments could include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

1-17. (canceled)

18. A method comprising:
obtaining, by a computing system, an observation of demographic values of an individual, comorbidity values of the individual, vital sign values of the individual, or blood test values of the individual, wherein the individual was diagnosed with portal hypertension or cirrhosis;
applying, by the computing system, a machine learning model to the observation, wherein the machine learning model was trained with a training data set, wherein the training data set contains observations of corresponding demographic values, comorbidity values, vital sign values, blood test values, or disease progression values for a plurality of individuals diagnosed with portal hypertension or cirrhosis, and wherein the machine learning model is configured to provide a prediction of: (i) a hazard ratio of whether the individual is expected to exhibit progression to a condition related to portal hypertension or cirrhosis, or (ii) a period of time between that of the observation and a further diagnosis of the condition; and
providing, by the computing system, the prediction based on the observation.

19. The method of claim 18, further comprising:
applying, by the computing system, a second machine learning model to the observation, wherein the second machine learning model was trained with at least part of the training data set, and wherein the second machine learning model is configured to provide a second prediction of: (i) a second hazard ratio of whether the individual is expected to exhibit progression to a second condition related to portal hypertension or cirrhosis, or (ii) a second period of time between that of the observation and a second further diagnosis of the second condition; and
providing, by the computing system, the second prediction based on the observation.

20. The method of claim 19, further comprising:
applying, by the computing system, a further machine learning model to the observation, wherein the further machine learning model was trained with at least part of the training data set, and wherein the further machine learning model is configured to provide a further prediction of: (i) a further hazard ratio of whether the individual is expected to exhibit progression to any condition related to portal hypertension or cirrhosis, and (ii) a further period of time between that of the observation and a further diagnosis of any condition related to portal hypertension or cirrhosis; and
providing, by the computing system, the further prediction based on the observation.

21. The method of claim 18, wherein providing the prediction comprises displaying the prediction on a graphical user interface.

22. The method of claim 18, wherein obtaining the observation comprises receiving the observation from a client device in communication with the computing system over a network, and wherein providing the prediction comprises transmitting the prediction to the client device.

23. The method of claim 18, wherein the disease progression values for a particular individual of the plurality of individuals includes an index date and one or more outcomes, and wherein each of the one or more outcomes indicates a particular condition and an observed period of time between its index date and when the particular condition was diagnosed.

24. The method of claim 23, wherein the disease progression values also include one or more additional outcomes, and wherein each of the one or more additional outcomes indicates an unknown condition and an additional observed period of time between the index date and when the unknown condition was identified.

25. The method of claim 23, wherein there is at least six months of vital sign values or blood test values prior to the index date in the disease progression values for the plurality of individuals.

26. The method of claim 23, wherein the particular condition is one of varices, variceal hemorrhages, recurrent variceal hemorrhages, ascites, refractory ascites, hepatic encephalopathy, recurrent hepatic encephalopathy, portosystemic shunts, or jaundice.

27. The method of claim 18, wherein the demographic values include ages, genders, races, or ethnicities of the plurality of individuals.

28. The method of claim 18, wherein the vital sign values include body mass indices, blood pressure readings, or heart rates of the plurality of individuals.

29. The method of claim 18, wherein the comorbidity values include indications of diabetes or obesity.

30. The method of claim 18, wherein values within the training data set are 20%-60% populated.

31. The method of claim 18, wherein the machine learning model is based on gradient boosting.

32. The method of claim 18, wherein the machine learning model is based on gradient boosting and survival time analysis.

33. The method of claim 18, wherein the training data set includes at least 10,000 observations gathered from medical claim records or electronic health records.

34. The method of claim 18, wherein the hazard ratio is provided as a Boolean indication of progression to the respective condition.

35. The method of claim 18, wherein the observations in the training data set also include indications of medications, prescriptions, or treatments relating to the plurality of individuals, and wherein the observation also includes indications of medications, prescriptions, or treatments relating to the individual.

36. An article of manufacture including a non-transitory computer-readable medium, having stored thereon program instructions that, upon execution by a computing system, cause the computing system to perform operations of comprising:
obtaining an observation of demographic values of an individual, comorbidity values of the individual, vital sign values of the individual, or blood test values of the individual, wherein the individual was diagnosed with portal hypertension or cirrhosis:
applying a machine learning model to the observation, wherein the machine learning model was trained with a training data set, wherein the training data set contains observations of corresponding demographic values, comorbidity values, vital sign values, blood test values, or disease progression values for a plurality of individuals diagnosed with portal hypertension or cirrhosis, and wherein the machine learning model is configured to provide a prediction of: (i) a hazard ratio of whether the individual is expected to exhibit progression to a condition related to portal hypertension or cirrhosis, or (ii) a period of time between that of the observation and a further diagnosis of the condition; and
providing the prediction based on the observation.

37. A computing system comprising:
one or more processors; and
memory containing program instructions that, upon execution by the one or more processors, cause the computing system to perform operations comprising:
obtaining an observation of demographic values of an individual, comorbidity values of the individual, vital sign values of the individual, or blood test values of the individual, wherein the individual was diagnosed with portal hypertension or cirrhosis;
applying a machine learning model to the observation, wherein the machine learning model was trained with a training data set, wherein the training data set contains observations of corresponding demographic values, comorbidity values, vital sign values, blood test values, or disease progression values for a plurality of individuals diagnosed with portal hypertension or cirrhosis, and wherein the machine learning model is configured to provide a prediction of: (i) a hazard ratio of whether the individual is expected to exhibit progression to a condition related to portal hypertension or cirrhosis, or (ii) a period of time between that of the observation and a further diagnosis of the condition; and
providing the prediction based on the observation.
